(19) United States
(12) Patent Application Publication
(10) Pub. No.: US 2025/0213108 A1
(43) Pub. Date: Jul. 3, 2025

(54) Methods and Systems for Detecting Risk for Development of a Retinal Condition Using Visual Field Tests

(71) Applicant: Notal Vision Ltd., Tel Aviv (IL)

(72) Inventors: Hanoch Gideon Benyamini, Ramat Hasharon (IL); Kester Nahen, Centreville, VA (US); Moshe Havilio, Tel Aviv (IL); Yair Alster, Tel Aviv (IL); Omer Rafaeli, Udim (IL)

(73) Assignee: Notal Vision Ltd., Tel Aviv (IL)

(21) Appl. No.: 19/084,971

(22) Filed: Mar. 20, 2025

Related U.S. Application Data
(63) Continuation of application No. PCT/IB2023/059577, filed on Sep. 27, 2023.
(60) Provisional application No. 63/411,044, filed on Sep. 28, 2022.

(51) Int. Cl. A61B 3/00; A61B 3/024; A61B 3/10; A61B 3/12; A61B 5/00; A61K 49/00; G16H 50/30

(52) U.S. Cl. CPC A61B 3/0025; A61B 3/024; A61B 3/102; A61B 3/1233; A61B 3/1241; A61K 49/0034; A61K 49/0043; G16H 50/30; A61B 5/7275

(57) ABSTRACT

Methods and systems for identifying a patient at increased risk for development of a retinal condition employ visual field tests. A method of identifying a patient at increased risk for development of a retinal condition includes monitoring the patient via one or more visual field tests. Each of the visual field tests includes displaying stimuli for viewing by an eye of the patient and receiving indications of locations of the stimuli as perceived by the patient. A visual field test score for each of the visual field tests is determined based on the stimuli and the indications of locations of the stimuli as perceived by the patient. In response to at least one of the visual field test scores exceeding a screening threshold, the patient is identified as being at increased risk for development of the retinal condition.

### CROSS REFERENCE TO RELATED APPLICATION DATA

The present application is a Continuation of PCT/IB2023/059577 filed Sep. 27, 2023; which claims the benefit of U.S. Provisional Application No. 63/411,044 filed Sep. 28, 2022, the disclosures which are incorporated herein by reference in their entirety for all purposes.

### BACKGROUND

Age-related macular degeneration (AMD) is the leading cause of blindness among people over the age of 50 in the western world. It is a bilateral, although asymmetric disease, and comes in two forms. Dry or non-neovascular AMD is the more common and milder form of AMD, accounting for 85-90% of all AMD. The key identifier for dry AMD is small, round, white-yellow lesions (also known as Drusen) in the macula. Vision loss associated with dry AMD is far less dramatic than in the case of wet AMD. There is currently no treatment available for dry AMD. It is estimated that as many as 14 million people suffer from dry AMD in the United States alone.

Wet AMD is less prevalent than the dry form, accounting for about 10-15% of AMD cases. The term “wet” denotes choroidal neovascularization (CNV), in which abnormal blood vessels first develop beneath the retinal pigment epithelium (RPE) layer of the retina, initially in a non-exudative stage, and subsequently progress to an exudative stage and leak fluid in spaces within and under the retina. Wet AMD is characterized by the development of choroidal angiogenesis, which causes severe, and potentially rapid, visual deterioration. Patients suffering from wet AMD often report symptoms of visual distortions. The visual distortion typically consists of perceiving straight lines as curved or discontinued due to deformation of the retina in a region overlying the choroidal angiogenesis. The wet form of AMD accounts for about 60% of all cases of adult blindness in the United States. In the U.S. alone there are 200,000 new cases of wet AMD every year and a total of 1.7 million blind people from AMD.

Treatment modalities for wet AMD may include laser photocoagulation, Photodynamic therapy (PDT) and intravitreal injections of anti-vascular endothelium growth factors (Anti-VEGF). Experimental treatments that are under current investigation include feeder vessel coagulation, trans-pupillary thermotherapy (TTT) and gene therapy. All these proven or experimental therapies may halt or slow progression of the disease only if detected at an early stage and will not reverse existing retinal damage. Therefore, early detection is crucial to prevent severe visual loss.

Since approximately 12% of dry AMD cases develop wet AMD and subsequent significant vision loss or blindness within 10 years, a patient diagnosed with dry AMD must be routinely examined by an ophthalmologist once or twice a year, depending on the severity of his condition. The patient is usually also given a so-called “Amsler grid” for weekly self-examination at home for symptoms of wet AMD. The patient is advised to consult an ophthalmologist immediately in the event that symptoms are noticed. The Amsler grid and its modifications (such as the “threshold Amsler” or the “red Amsler”) have been shown to be poor detectors of early changes associated with wet AMD for several reasons. One reason is the phenomenon of “filling-in” whereby the brain fills in missing parts in the pattern or corrects defects or distortions in the pattern. The subject thus fails to perceive a distorted pattern as being distorted. Another problem with the Amsler grid is the inability of patients to adequately fixate their vision on a fixed point while taking the test. The Amsler test also suffers from low compliance stemming from the non-interactive nature of the test.

The degree of visual deterioration is a function of the size of the lesion and its proximity to the fovea at the time of diagnosis. Although most lesions probably start outside the foveal area, 70% are already foveal and large (>1500 microns) at the time of diagnosis. It is therefore crucial to identify the lesions at the earliest possible stage, while they are still small and have not reached the fovea. It is known that the progression of the disease is relatively rapid. As many as 70-80% of patients with wet AMD are already experiencing significant vision loss when they first consult their ophthalmologist because the disease has progressed considerably. This is due to the poor validity of existing self-assessment methods for detecting an AMD-related lesion at an early stage, and the time elapsed between noticing the symptoms and seeing an ophthalmologist.

A reliable method for diagnosing non-exudative neovascularization and wet AMD at the earliest possible stage, in conjunction with a referral system aimed at lowering the incidence of visual deterioration in this devastating disease, are imperative. If detected early, treatment may prevent additional vision loss. It is therefore crucial to detect the transition from dry to wet AMD as early as possible.

### BRIEF SUMMARY

The following presents a simplified summary of some embodiments of the invention to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

Methods and systems for identifying a patient at increased risk for development of a retinal condition, such wet (exudative neovascular) AMD, retinal atrophy, central and non-central Geographic Atrophy (GA) in a patient employ visual field tests to monitor the patient. In response to a visual field test score exceeding a threshold, the patient is identified as having an increased risk for developing the retinal condition relative to a selected population. The patient can be subsequently monitored for development of the retinal condition using an increased frequency and/or imaging examinations (e.g., optical coherence tomography (OCT) imaging of the retina, optical coherence tomography angiography (OCTA) imaging of the retina, dye based angiography, namely fluorescein angiography (FA) and indocyanine green angiography (ICGA)). By using visual field tests to monitor for increased risk for developing the retinal condition, the effort and expense for monitoring the patient for developing the retinal condition can be tailored to the risk of the patient for developing the retinal condition.

Thus, in one aspect, a method of identifying a patient at increased risk for development of a retinal condition of a retina of the patient includes monitoring the patient via one or more visual field tests. Each of the visual field tests includes displaying stimuli for viewing by an eye of the patient and receiving indications of locations of the stimuli as perceived by the patient. A visual field test score is determined for each of the visual field tests based on the stimuli and the indications of locations of the stimuli as perceived by the patient. In response to at least one of the visual field test scores exceeding a screening threshold, the patient is identified as being at increased risk for development of the retinal condition relative to a selected population. In many embodiments, each of the one or more visual field tests measures visual hyperacuity.

The method can be applied to any suitable retinal condition. For example, the retinal condition can include any one or combination of wet age-related macular degeneration (AMD), retinal atrophy, central geographic atrophy, or non-central geographic atrophy.

The method can further include monitoring the patient via at least one imaging examination of the retina in response to the at least one of the visual field test scores exceeding a screening threshold. For example, the at least one imaging examination can include any one or a combination of an optical coherence tomography (OCT) examination of the retina, an optical coherence tomography angiography (OCTA) examination of the retina for development of non-exudative neovascularization, or a dye-based angiography examination (e.g., fluorescein angiography, indocyanine green angiography). In many embodiments, the development of the retinal condition is not detected during a subsequent examination of the at least one imaging examination of the retina. The at least one imaging examination of the retina can include a sequence of imaging examinations of the retina. The one or more visual field tests can be conducted at a first frequency. The at least one imaging examination can include a sequence of imaging examinations conducted at a second frequency that is greater than the first frequency. In some embodiments, the second frequency is at least twice the first frequency.

In many embodiments of the method, the patient is identified as having the increased risk prior detectability of the retinal condition. For example, in many embodiments of the method, development of the retinal condition is not detected within one month of one of the visual field test scores exceeding the screening threshold.

In many embodiments of the method, the screening threshold is selected so that the patient has a substantial increase in risk for developing the retinal condition relative to the selected population. For example, the screening threshold can be selected so that the risk for developing the retinal condition relative to the selected population is at least 50 percent higher, at least 100 percent higher, or at least 200 percent higher. The increased risk for development of the retinal condition can be for any suitable time frame from the exceedance of the screening threshold (e.g., any suitable time frame between 1 month and 60 months).

The selected population can already have a higher risk for developing the retinal condition. For example, a contra-lateral eye of the patient and an eye of each of the selected population can be afflicted with wet AMD.

In another aspect, a system for identifying a patient at increased risk for development of a retinal condition of a retina of the patient includes a server. The server includes at least one processor and instructions. The instructions are executable by the at least one processor to: (1) receive visual field test data generated via one or more visual field tests of the patient, wherein each of the visual field tests comprises displaying stimuli for viewing by an eye of the patient and receiving indications of locations of the stimuli as perceived by the patient, wherein a visual field test score is determined for each of the one or more visual field tests based on the stimuli and the indications of locations of the stimuli as perceived by the patient; and (2) in response to at least one of the visual field test scores exceeding a screening threshold, generate an output that identifies the patient as being at increased risk for development of the retinal condition relative to a selected population. In many embodiments, each of the one or more visual field tests measures visual hyperacuity.

The system can be applied to any suitable retinal condition. For example, the retinal condition can include any one or combination of wet age-related macular degeneration (AMD), retinal atrophy, central geographic atrophy, or non-central geographic atrophy.

The system can further be configured to cause monitoring the patient via at least one imaging examination of the retina in response to the at least one of the visual field test scores exceeding a screening threshold. For example, the at least one imaging examination can include any one or a combination of an optical coherence tomography (OCT) examination of the retina, an optical coherence tomography angiography (OCTA) examination of the retina for development of non-exudative neovascularization, or a dye-based angiography examination (e.g., fluorescein angiography, indocyanine green angiography). In many embodiments, the development of the retinal condition is not detected during a subsequent examination of the at least one imaging examination of the retina. The at least one imaging examination of the retina can include a sequence of imaging examinations of the retina. The one or more visual field tests can be conducted at a first frequency. The at least one imaging examination can include a sequence of imaging examinations conducted at a second frequency that is greater than the first frequency. In some embodiments, the second frequency is at least twice the first frequency.

In many embodiments of the system the patient is identified as having the increased risk prior detectability of the retinal condition. For example, in many embodiments of the system, development of the retinal condition is not detected within one month of one of the visual field test scores exceeding the screening threshold.

In many embodiments of the system, the screening threshold is selected so that the patient has a substantial increase in risk for developing the retinal condition relative to the selected population. For example, the screening threshold can be selected so that the risk for developing the retinal condition relative to the selected population is at least 50 percent higher, at least 100 percent higher, or at least 200 percent higher. The increased risk for development of the retinal condition can be for any suitable time frame from the exceedance of the screening threshold (e.g., any suitable time frame between 1 month and 60 months).

The selected population can already have a higher risk for developing the retinal condition. For example, a contra-lateral eye of the patient and an eye of each of the selected population can be afflicted with wet AMD.

For a fuller understanding of the nature and advantages of the present invention, reference should be made to the ensuing detailed description and accompanying drawings.

### BRIEF DESCRIPTION OF THE DRAWINGS

FIG. 1 is a simplified schematic flow chart diagram of a method of identifying a patient at increased risk for development of a retinal condition, in accordance with many embodiments.

FIG. 2 is a schematic diagram of a system for accomplishing visual field tests to detect increased risk for development of a retinal condition, in accordance with many embodiments.

FIG. 3 is a schematic diagram of a device for accomplishing visual field tests to detect increased risk for development of a retinal condition, in accordance with many embodiments.

FIGS. 4A and 4B are simplified schematic flow chart diagram of a method of accomplishing visual field tests to detect increased risk for development of a retinal condition, in accordance with many embodiments.

FIG. 5 schematically illustrates selected exemplary screen representations including exemplary test patterns that may be presented to a tested subject and selected schematic representations of how the test patterns may be perceived by a tested subject in selected stages of the testing.

FIG. 6 shows a plot of example visual field test scores for a monitored subject.

FIG. 7 is a simplified schematic flow chart diagram of a method for mapping an eye using visual field tests to detect increased risk for development of a retinal condition, in accordance with embodiments.

FIG. 8 is a schematic illustration of an example pattern displayed to a subject in the method of FIG. 7.

FIG. 9 is shows an example metamorphopsia map of an eye generated using the method of FIG. 7.

FIG. 10 is a simplified schematic flow chart diagram of a method of determining a screening threshold for use in the method of FIG. 1.

FIG. 11 shows Kaplan-Meier estimated conversion functions for population subsets that illustrate efficacy of the method of FIG. 1.

### DETAILED DESCRIPTION

In the following description, various embodiments of the present invention will be described. For purposes of explanation, specific configurations and details are set forth in order to provide a thorough understanding of the embodiments. However, it will also be apparent to one skilled in the art that the present invention may be practiced without the specific details. Furthermore, well-known features may be omitted or simplified in order not to obscure the embodiment being described.

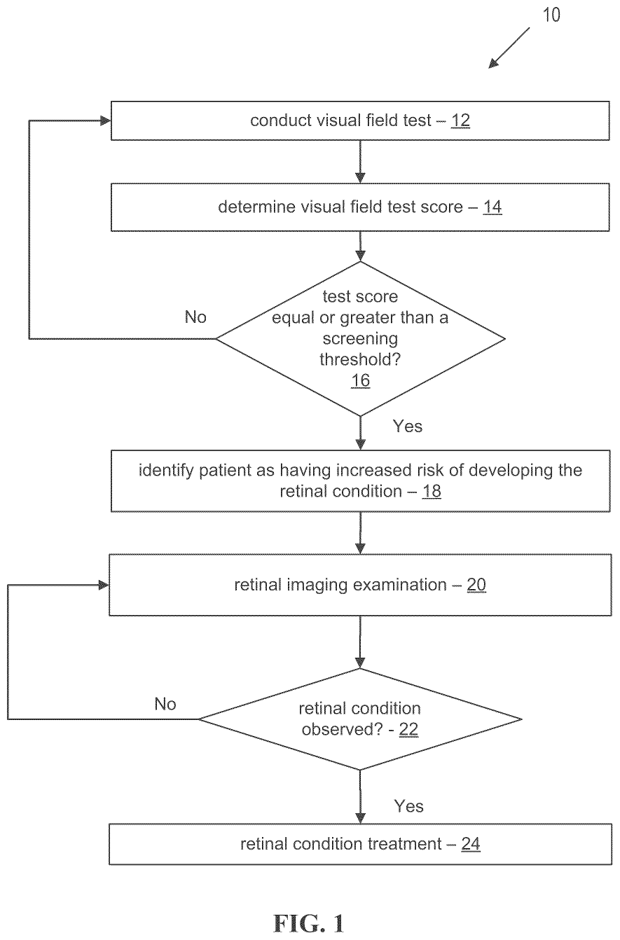

Turning now the drawing figures in which similar reference-identifiers refer to similar features, FIG. 1 shows a simplified schematic flow chart diagram of a method 10 of identifying a patient at increased risk for development of a retinal condition (e.g., wet (exudative neovascular) AMD, retinal atrophy, central geographic atrophy, and/or non-central geographic atrophy), in accordance with many embodiments. In many implementations, the method 10 employs visual field tests, which may be followed by a sequence of retinal imaging examinations. The visual field tests are employed until the patient has a visual field test score indicative of a sufficient increase in risk for development of the retinal condition, which may result in switching to monitoring the patient via one or more retinal imaging examinations.

In act 12, the patient is subjected to a visual field test. In many embodiments, the visual field test includes displaying stimuli for viewing via the eye having the retina and receiving indications of locations of the stimuli as perceived by the patient. In act 14, a visual field test score is determined based on the stimuli and the indications of locations of the stimuli as perceived by the patient. Any suitable approach, such as the methods 300, 600 described herein, can be used to accomplish act 12 and act 14. In act 16, if the visual test score is less than a screening threshold, the method 10 continues with monitoring the patient via the visual field tests.

In act 16, if the visual field test score is equal to or greater than the screening threshold, the method 10 proceeds to act 18 in which the patient is identified as having an increased risk for development of the retinal condition. After the patient is identified as having an increased risk for development of the retinal condition, any suitable course of action can then be pursued. In some embodiments, the retina is then monitored using one or more imaging examinations. Any suitable imaging examination of the retina can be employed. For example, the retina can be monitored using one or more of an optical coherence tomography (OCT) imaging examination, an optical coherence tomography angiography (OCTA) imaging examination, or a dye-based angiography examination of the retina (act 20). In act 22, if the retinal condition is not observed via the imaging examination of the retina conducted in act 20, the method 10 can continues with monitoring the patient via retinal imaging examinations. In act 22, if the retinal condition is observed, the method 10 can proceeds to act 24 in which the retinal condition is treated. In many instances, the treatment of the retinal condition in act 24 includes a sequence of monitoring retinal imaging examinations along with any suitable therapeutic treatment, such as one or more injections of a therapeutic compound into the eye and/or laser eye surgery.

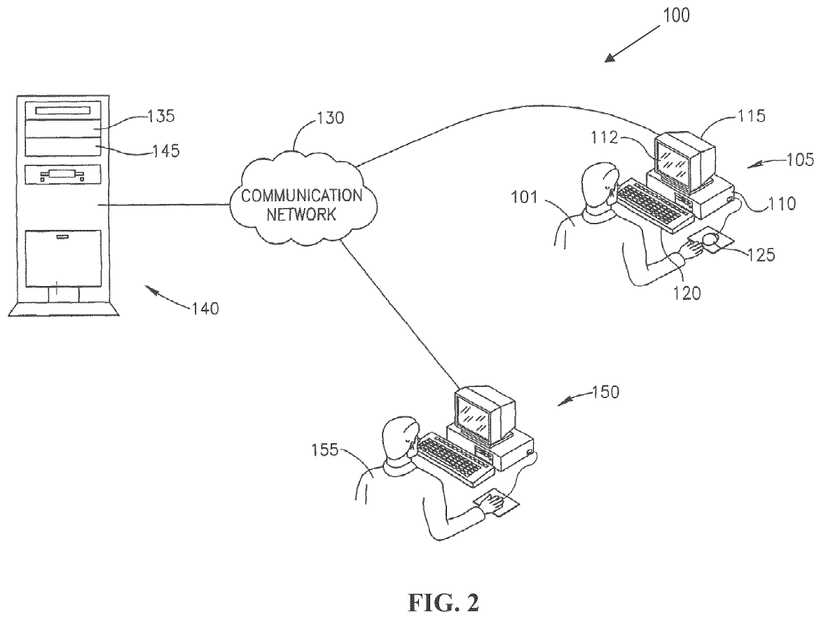

FIG. 2 is a schematic diagram of a system 100 for accomplishing visual field tests to detect increased risk for development of a retinal condition (e.g., non-exudative neovascularization, wet (exudative neovascular) AMD, retinal atrophy, central geographic atrophy, and/or non-central geographic atrophy), in accordance with many embodiments. A subject 101 performs an eye test using a computer system 105. The computer system 105 may comprise a computer 110, a display device 115 having a screen 112 and one or more computer input devices such as a keyboard 120 or a computer mouse 125. The computer system 105 may communicate over a communication network schematically indicated by the cloud labeled 130. The network 130 may be, for example, the Internet, a local area network (LAN), a wide area network (WAN), an Intranet, a private area network (PAN), virtual networks implemented over the Internet, other private and/or commercial communication networks, or any other suitable type of communication network known in the art.

A processor 135 in a network server 140 stores data relating to execution of an eye test to be performed by the subject 101 to be described in detail below. The eye test is communicated from the server 140 to the subject's computer 110 over the network 130. The subject 101 inputs responses to the eye test using one or more of the computer input devices such as the keyboard 120 or the mouse 125. The subject's responses are communicated over the network 130 to the processor 135 and stored in the memory 145. The processor 135 is configured to analyze the subject's response, to make a diagnosis of the subject's conditions and to recommend future follow-up or recommend prompt examination, all in real time, for the subject. The diagnosis and recommendation may be communicated over the network 130 to the subject's computer system 105 and/or to a terminal 150 of a health care provider 155. The processor 135 is also configured to store in the memory 145 dates on which the subject is to perform an eye test executed by the processor 135. If, for example, the subject 101 has been instructed by the health and provider 155 to perform the test once per week, the processor 135 may send a message over the communication network 130 when 10 days have elapsed since the last time he took the test, informing the subject of his failure to take the test as instructed. A similar message may be sent to the health care provider 155. A responsible individual may be designated, in such a case, to contact the subject 101, for example, by telephone to clarify why the subject 101 has not performed the test as instructed and to impress upon the subject the importance of performing the test as indicated.

### “Moving Pattern” Test Method

A method disclosed hereinbelow is based on the presentation of a first pattern at a first location on the surface of a display device (such as, but not limited to the screen 112 of the display device 115) to the patient or the test subject. After the patient has fixated on a fixation target presented on or adjacent to the first pattern, the first pattern disappears from the first location of the display device and a second pattern is presented at a second location on the display device. The second pattern may be identical to the first pattern (except for the fact that it appears at a different location on the display device), or it may be different from the first pattern by having one or more portions thereof changed or altered. Such changes or alterations may include distortion of the shape or elimination of one or more portions of the first pattern, or changes in the color or appearance of one or more portions of the first pattern. Because the first pattern is made to disappear from the first location on the display device and the second pattern appears at a second location of the display device different than the first location, the patient or test subject may visually perceive this as a movement or jump of the pattern from the first location to the second location on the display device, in other words, the patient may perceive a pattern “jumping” on the screen of the display device from a first to a second location even though the pattern does not actually move on the display device (the pattern actually disappears from a first location on the display device and appears at a second location on the display device). This is why this particular embodiment of the testing method is referred to as the “moving pattern” or “jumping pattern” test hereinafter.

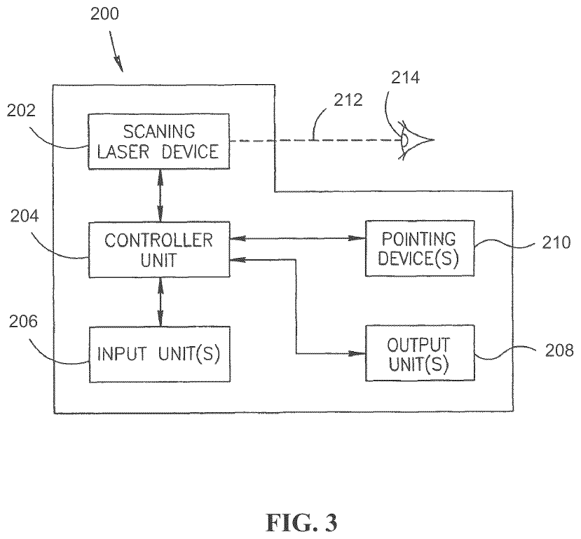

FIG. 3 is a schematic diagram of a system 200 for accomplishing visual field tests to detect increased risk for development of a retinal condition (e.g., non-exudative neovascularization, wet (exudative neovascular) AMD, retinal atrophy, central geographic atrophy, and/or non-central geographic atrophy), in accordance with many embodiments. In the system 200, the images of the test patterns and fixation target(s), and possibly the log-on screen(s) may be directly projected on the retina of an eye 214 of the test subject (not shown) by suitably directing a laser beam 212 (schematically represented by the dashed line labeled 212) through the pupil of the tested eye 214 and by suitably scanning the laser beam 214 across the retinal surface to form projected images of the test patterns and/or fixation target(s) at specified locations on the retinal surface. The system 200 may include a scanning laser device 202. The scanning laser device 202 may be a scanning laser ophthalmoscope (SLO) device as is known in the art. Or any other device capable of controllably scanning a beam of coherent or non-coherent light across the retina of an eye. The scanning laser device 202 may be suitably coupled to a controller unit 204 or to a computer (not shown) for controlling the operation of the scanning laser device 202. The controller unit 204 may also be a computer such as a workstation, or mainframe, or laptop computer or a hand-held or other portable computing device, or a personal computer or any other type of computing device known in the art. The controller unit 204 may be coupled to suitable pointing device(s) 210. The pointing device(s) 210 may be a mouse (not shown), and/or keyboard connected to a computer or may be any other suitable pointing device or devices as disclosed hereinabove or as known in the art. The system 200 may also include one or more output unit(s) 208, such as, but not limited to a display, a printer unit, or any other suitable output device for enabling interaction of a user with the system 200 and/or for producing hard copy output of test results or the like. The output unit(s) 208 may be suitably coupled or connected to the controller unit 204.

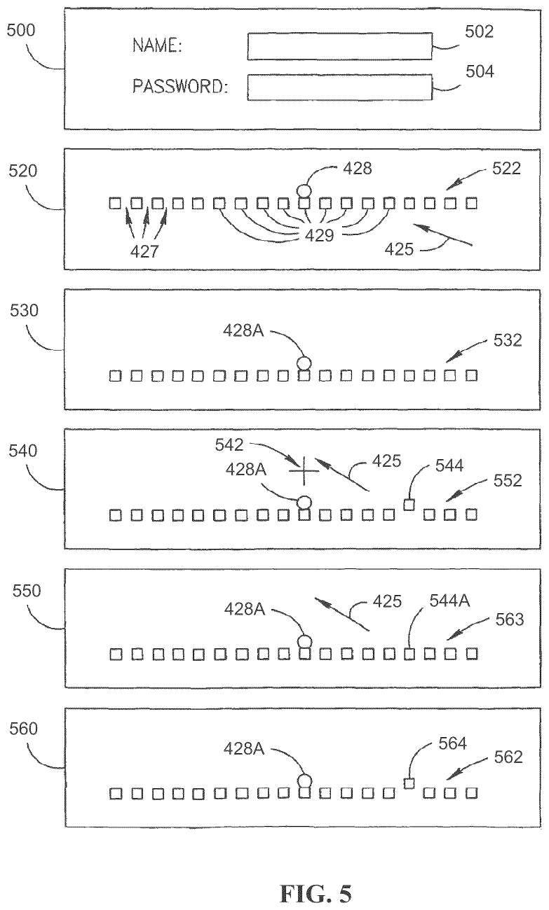

In operation the system 200 may be used for applying any of the tests disclosed hereinabove but instead of showing the test pattern, and the fixation targets on a screen 112 of a display device 115, the images of the test patterns and the fixation target(s) may be directly projected onto the retina of the tested eye 214 by the scanning laser device 202 by suitably scanning the laser beam 212 on the retina of the eye 214. The laser beam 212 may also be used to project an image of a cursor (similar to the cursor 425 of FIG. 5) directly on the retina of the eye. The movement of such a projected cursor may be controlled by the one or more of the pointing devices 210, such as but not limited to a mouse (not shown). The system 200 may be used to administer to a patient any of the tests disclosed hereinabove (including but not limited to the moving line test and the flash test) and to record and star the responses of the patient including but not limited to the marking of parts or portions or segments at which distortions or modifications as disclosed herein above were perceived and marked by the patient. The system 200 may also process the test results using any of the methods and test criteria disclosed herein above to produce a positive or negative diagnosis. The system 200 may also be suitably connected to a communication network (such as, but not limited to the communication network 130 of FIG. 2) and may communicate with other devices or computers, or the like over the communication network.

It is noted that the laser scanning device 202 may be replaced or substituted with other scanning devices (not shown) known in the art which are capable of directing a narrow light beam having a suitably narrow beam cross-sectional area onto an eye and scanning the beam controllably across the retina. The light beam need not be a laser beam but may be any beam of non-coherent light which may be suitably scanned across a retina with sufficient speed and resolution.

It is noted that the construction and operation of laser scanning ophthalmoscopy devices is well known in the art, is not the subject matter of the present invention and is therefore not described in detail herein.

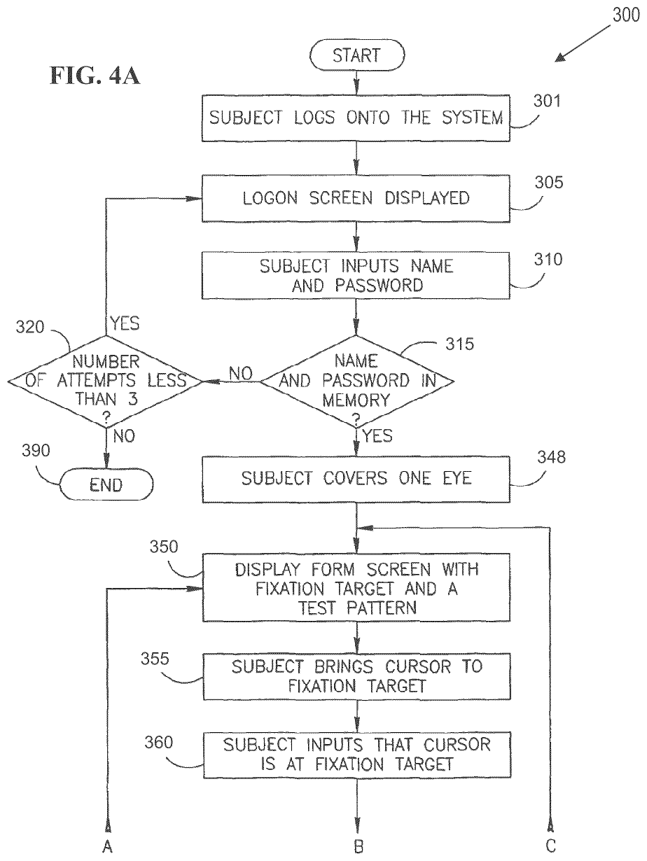

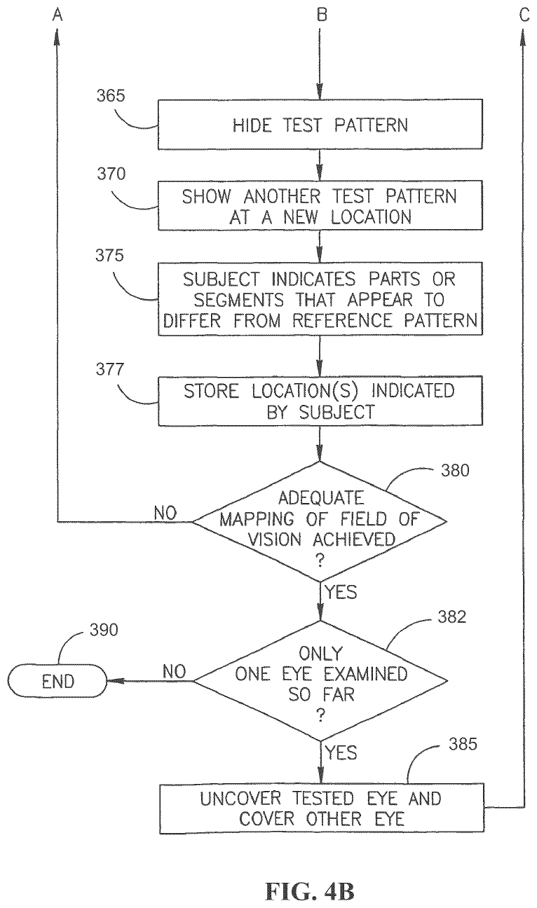

FIGS. 4A and 4B are simplified schematic flow chart diagram of a method 300 of accomplishing visual field tests to detect increased risk for development of a retinal condition (e.g., non-exudative neovascularization, wet (exudative neovascular) AMD, retinal atrophy, central geographic atrophy, and/or non-central geographic atrophy), in accordance with many embodiments. FIG. 5 schematically illustrates selected exemplary screen representations including exemplary test patterns which may be presented to a tested subject, in an exemplary embodiment of the method 300 and selected schematic representations of how the test patterns may be perceived by a tested subject in selected stages of the testing. Additional details of the method 300 is described in U.S. Pat. No. 7,275,830, which is hereby incorporated herein by reference in its entirety.

FIG. 5 includes schematic screen diagrams 500, 520, 530 and 560 (also referred to as screens 500, 520, 530 and 560 hereinafter) which schematically illustrate the patterns displayed on the screen 112 of the subject's display device 115 at various different exemplary steps of the eye test performed by the system illustrated in FIG. 2, and schematic screen diagrams 540 and 550 (also referred to as screens 540, and 550 hereinafter) that schematically illustrate the possible appearance of the screen 112 as may be perceived by the tested subject at some exemplary steps of the eye test. It is noted that screen 500 of FIG. 5, which does not include test patterns, schematically represents a possible log-on screen that may be presented to the test subject.

It is noted that the exemplary schematic screens 500, 520, 530, 540, 550, and 560 of FIG. 5 are schematically drawn for illustrative purposes only and are not drawn to scale. Additionally, the sizes of the various patterns, pattern segments, fixation targets, are not drawn to scale, and their sizes and their relation to the screen size are arbitrarily shown for illustrative purposes only.

Turning to FIG. 4A and FIG. 4B, in step 301, the subject 101 may log onto the computer system 105. The processor 135 may cause log-on screen 500 to be displayed on the subject's display device 115 (step 305). The log-on screen 500 prompts the subject to input his name into a field 502 and to input a previously assigned password into a field 504 for accessing the processor 135. In step 310 the subject inputs his name and password using computer input devices such as the keyboard 120 or the mouse 125. The processor 135 then checks whether the inputted name and password are stored in the memory 145 (step 315). If the inputted name and password do not match a corresponding name and password that are stored in the memory 145, the processor 135 determines whether the number of attempts the subject has made to input a name and password is less than a predetermined number of attempts such as, for example, three attempts (step 320). If yes, the process returns to step 305. If no, the process terminates.

If at step 315 the processor determines that the name and password are in the memory 145, the process continues in step 348 by the subject being instructed to cover an eye, so that the test is performed using one eye only. The subject may be instructed to cover an eye by displaying appropriate text (not shown) or a drawing (not shown) or an icon (not shown), or a graphic element (not shown) or any combination thereof on the screen of the display device 115.

It is noted that the tested subject 101 may be asked to cover a specific eye (for example, the subject may be asked to cover the right eye and to view the screen 112 with the uncovered left eye for testing the left eye). In this way the computer system 110 may automatically record that the left eye is being tested. Alternatively, the tested subject 101 may be asked to mark or input or otherwise indicate which eye is to be tested, such as, for example, by clicking a cursor on one of two boxes (not shown) which may be presented on the log-on screen 500 or on any other suitable screen presented to the subject before the test begins. In such a case the test results may be labeled as taken from the eye selected by the subject 101.

In step 350 a form screen 520 is displayed in which a pattern such as the segmented line 522 is displayed. This is by way of example only, and other suitable patterns may be used. The pattern may comprise a single component or may comprise several components which may or may not be all identical. Thus, the pattern may comprise several lines, one or more circles, lines and circles together, or any other suitable combination of pattern elements, including but not limited to one or more straight lines, dotted lines, curved lines, linear or nonlinear segments, dots, and other various geometrical patterns such as but not limited to circles, arcs, rectangles, squares, triangles, and the like. The screen 112 of the display device 115 may display a visually noisy background to the dis-played pattern. The line 522 may be composed of several short segments 429 separated by gaps 427. Alternatively, the displayed line may be continuous (not shown).

Preferably, the length of the line 522 is such that when the tested subject's eye is at a distance of approximately 50 centimeters from the screen 112 of the display device 115 the length of the line 522 corresponds to a cone angle of 1 to 20 degrees. It is noted that while in most tests the length of the lines used corresponded to a cone angle of 14 degrees, other different line lengths may be used. At these viewing conditions, each of the gaps 427 between the segments 429 (the distances separating two adjacent segments 429) may correspond to a cone angle of between 1 minute arc to about 2 degrees. Other, different line lengths and gap sizes may however also be used. For example, if the test pattern is a continuous line, there are no gaps. If test patterns in which a continuous line is used, no segments are used and there are therefore no gaps.

If the pattern consists of two or more parallel lines, the spacing between the lines corresponds, preferably, to a cone angle from about 10 to about 600 minutes arc. The test patterns may be horizontal patterns such as, but not limited to the horizontal line 522 illustrated in screen 500 but may also be vertical patterns such as but not limited to a vertical segmented line (not shown) or slanted patterns such as but not limited to a slanted segmented line (not shown). A fixation target 428 may be displayed on the screen adjacent to one of the segments 429. The fixation target may be a circular pattern such as the circular fixation target 428 of screen 520 of FIG. 5 or may have any other shape or pattern suitable for serving as a fixation target for focus the tested eye thereon, such as but not limited to a square pattern, a triangular pattern, or any other suitable pattern which is suitable for functioning as a fixation target.

While the fixation target 428 illustrated in FIG. 5 is a circular pattern which appears close to the middle segment of the line 522, other different forms of the fixation target may be used. For example, the fixation target may be implemented as a hollow (unfilled) circle (not shown) surrounding the middle segment of the line 522 or superimposed thereon, or as any other suitable pattern which is positioned close to or is superimposed upon the line 522.

Generally, the shape of the fixation target may depend, inter alia, on the shape and dimensions of the test pattern that is being used in the test. The fixation target may have the same color of the test pattern (such as, for example, the segmented line 522) or may have a different color than the color of the test pattern. In accordance with another variation, the fixation target may be the central (middle) segment of the line 522, in which case the middle segment may or may not have a color that is different than the color of the remaining segments 429 of the line 522 in order to make easily identifiable by the test subject.

In the example in which a single segmented line serves as the test pattern, the subject may be instructed to bring a cursor 425 appearing on the screen to the fixation target 428. In order to aid the subject, the movement of the cursor 425 may be restricted to a line (not shown) which is parallel to the line 522 so that the cursor 425 always points to one of the segments 429.

The subject 101 may be asked (for example, by an instructor, a physician an ophthalmologist or any other person training the subject in performing the test) or otherwise instructed (such as for example by displaying appropriate messages or text on the screen 112 of the display device 115) to point the cursor 425 at the fixation target 428. The subject 101 may perform this pointing in step 355 by using a computer device such as the keyboard 120, or more preferably the mouse 125. Other pointing devices may also be used for pointing, such as but not limited to, a digitizing tablet in conjunction with a stylus, a finger, a light pen in conjunction with a touch screen, or any other suitable pointing device or suitable input device known in the art.

The fixation target 428 may be sized so that it is large enough to be seen by the patient or test subject but small enough so that bringing the cursor 425 to the fixation target 428 is a demanding task for the test subject. This causes the subject to fixate his vision on the fixation target 428. Upon bringing the cursor to the segment 429, the subject may provide a suitable indication that he has positioned the cursor 425 to point at the fixation target 428. For example, the patient or test subject 101 may provide the indication by clicking on the mouse 125 or by depressing a predetermined key on the keyboard 120 (step 360). This input may serve as an indication or a verification that visual fixation has been achieved. It is noted that the size of the fixation target 428 may depend, inter alia, on the distance of the tested eye from the screen 112.

If the subject is using a pointing device and/or an input device which is different than the mouse 125 or the keyboard 120, the subject may indicate fixation on the fixation target 428 by performing any other suitable action. For example, if a touch screen (not shown) is used as an input device, the subject may touch the touch screen (not shown) with a light pen (not shown), or with a stylus (not shown) or with a finger (not shown) at the position at which the fixation target is displayed. Other suitable forms of indicating or confirming fixation may be used, depending, inter alia, on the input device or pointing device which is being used.

When the subject signals (for example, by clicking a button on the mouse 125, or by any other suitable way) that the cursor 425 is positioned to point at the fixation target 428, indicating that his vision is fixated on the fixation target 428, the line 522 is made to disappear from the screen 520 (step 365). After a predetermined delay time interval (for example a delay interval in the range of 0 to 200 milliseconds), a second pattern such as the segmented line 532 is made to appear (displayed) on the screen 112 at a location different than the location of the line 522 as shown in screen 530 (step 370) so as to allow the subject to form a perceived image of the segmented line 532. In this example, the segmented line 532 is similar to the line 522 but appears on the screen 112 at a location which is different than the location of the line 522.

It is noted that if the duration of the delay time interval is zero, the line 532 is presented on the screen 112 immediately after (or within the short time required by the computer 100 to process the subject's input and display the line 532 on the screen 112) the subject 101 indicated fixation. In most of the experimental eye tests conducted in patients no delay was used (the delay time interval was zero).

The line 532 may, for example, be parallel to the line 522. Since the subject's vision had been fixated on the fixation target 428, the line 532 will appear in the periphery of the subject's field of vision. Any disturbance in his vision due to a retinal lesion (such as but not limited to a lesion caused by AMD or diabetes or by other different pathological eye conditions) may be apparent to the test subject as a difference between the perceived image of the second pattern and a pre-defined reference pattern, which in this example is provided by the first pattern (the segmented straight line 522).

Additionally, or alternatively, the tested patient or subject 101 may have been told by a trainer (such as, for example, by an ophthalmologist or other medical or paramedical personnel) before the beginning of the test that he or she is going to be presented with test patterns which will look like a segmented straight line. In such a case, the subject 101 may conceive a “virtual” predetermined reference pattern which in this particular example of the test is a conceived image of a straight segmented line. The word “virtual” is used herein to indicate that the predetermined reference pattern is mentally conceived by the patient or test subject without having to actually present the patient with the test pattern. In other words the understanding of the patient of how the reference pattern (such as, for example, the straight segmented line of the example illustrated in FIG. 5) is supposed to look like may be based on the previous visual experience of the patient or test subject.

The explanation to the patient of what the reference pattern is going to look like may be advantageous, since in a small percentage of patients it may happen that in the first presentation of the first test pattern (such as for example in the initial presentation of the line 522) the image of the test pattern may fall on a lesioned retinal region. In such a case, the perceived image of the test pattern may be distorted. Therefore, in such a case, the perceived image of the initially presented line 522 is not usable as a reference pattern and the patient may see or detect a difference between the perceived image of the line 522 and the (virtual) reference image which the patient has been told to expect.

The difference between the perceived image of any of the test patterns (including, but not limited to, the lines 522 and 532) that may be presented to the patient and the reference pattern may be perceived by the test subject 101 in various different ways. Thus, as the line 522 is perceived by the subject to jump or move to the new location on the screen 112 one or more of the segments 429 of the line may seem to the subject not to arrive at their new position on the line 532 (of the screen 530) at exactly the same pace or contour as the other segments. In other words, one or more portions or segments of the line may temporarily seem to lag or to move differently relative to the other parts or segments of the perceived line. This may also be perceived by the subject as if one or more portions of the perceived line were wavy or moved or bulged for a short while or as if one or more of the segments or line portions deviated from the reference pattern (which is a straight segmented line, in the exemplary and non-limiting example of FIG. 5) before assuming again the perceived appearance of the reference pattern. Additionally or alternatively, depending, inter alia, on the nature of the retinal lesion present, one or more portions or segments of the perceived image of the test pattern (such as, for example, one or more the segments 429 of the perceived line 522) may appear to temporarily change their apparent brightness (such as becoming brighter or becoming darker), or change their color temporarily as the line moves or jumps, and then return to their originally perceived brightness or originally perceived color, respectively. Additionally or alternatively, one or more of the segments 429 or portions of the test pattern may appear to momentarily or temporarily become blurred or smeared.

Additionally, various different combinations of this differences may also be perceived by the subject. For example, one or more of the segments or portions of the test pattern may appear to lag or move differently than the other segments or portions of the pattern and also to change their perceived brightness. Other different combinations of differences may also be perceived by some patients.

For example, when the subject's vision is fixated at the location where the fixation target 428 had previously appeared (represented by the crossed lines 542 in screen 540), a segment 544 of the line 532 may appear to be out of line with other segments in the line 532 or may be otherwise distorted, blurred, shifted or discolored. It is noted that the screen labeled 540 of FIG. 5 represents the screen 112 and the pattern 532 as perceived by the tested subject. In other words, the illustrated line 532 with the shifted segment 544 are shown as perceived by a subject or patient having a retinal lesion and not as actually displayed on the screen 112. Thus, while the screen 530 of FIG. 5 schematically represents the image as actually presented to the subject 105 on the screen 112, the screen 540 of FIG. 5 represents the image perceived by a subject having a retinal lesion (in the tested eye) after the line 532 of screen 520 is made to disappear (hidden) and the line 532 of screen 530 is presented (or displayed) to the subject at a different location on the screen 112.

Screen 540 (of FIG. 5) illustrates a possible appearance of the line 532 (of screen 530) to an individual having a retinal lesion. The perceived line 552 of the perceived screen 540 represents the image of the line 532 presented in screen 530 as it may be perceived by an individual having a retinal lesion. The segment 542 of the perceived line 552 may typically be temporarily perceived as being out of line with other segments in the line 552. This is by way of example only, and other differences between the perceived image and the pre-defined reference pattern may be perceived, such as, but not limited to, one or more segments in the second pattern appearing to the subject as being shifted or wavy or lagging behind, or blurred, or dimming, or smeared, or bent, or otherwise distorted, or discolored. Additionally, one or more of the segments in the second pattern may be perceived by the subject to disappear or to be missing from the second pattern. As the subject subsequently shifts his vision from the fixation target 428 to the presented line 532, the segment 544 in the image of the perceived line 552 may appear to move into alignment with other segments in the line 532 as shown in screen 550. Thus, the line 563 of screen 550 (FIG. 5) schematically represents the perceived image of the line 522 as possibly perceived by the subject having a retinal lesion after the subject re-fixates his vision on the line 532. Thus, the segment 544A schematically represents the new perceived position of the previously perceived segment 544 after the patient shifted his eye to re-fixate on the new position of the line 532. The perceived segment 544A may now be perceived as realigned again with the rest of the perceived segments of the perceived line 563.

Typically, the reason for the presented line 532 being perceived as straight again (as illustrated in the perceived line 563 of screen 550) after the patient re-fixated his vision at the new position at which the line 532 appeared after the line 522 disappeared from the screen, is that in most cases when the subject shifts his vision from the fixation point 428 to the new location on the screen at which the line 532 appeared, after a certain time (typically a few hundred milliseconds or longer) the “filling-in” phenomenon disclosed hereinabove may occur.

The subject, in step 375, may indicate which, if any, of the segments in the line 532 appeared different or were perceived to behave differently than corresponding segments in the predefined reference pattern. This may be done by the subject bringing the cursor 425 to the segment or segments that appeared to move or to blur or to distort or to disappear, or to otherwise change (the segment 544 in this example) and clicking a button on the mouse 125 or a key on the keyboard 120, or by otherwise performing an action with a pointing device (not shown) or any other suitable input device. The data representing the location(s) on the screen 112 of the segment or segments in the region pointed to by the subject may thus be stored by the system (step 377) in the memory of the computer system 105, and/or in the memory 145 of the server 140 or by any other suitable storage means, such as but not limited to, a fixed or removable magnetic media storage device (Hard disc drive or floppy disc drive), optical storage device, magneto-optical storage device, holographic storage device or any other suitable storage device known in the art. This stored data may be used to locate and/or report and/or display and/or symbolically represent (in hard copy or otherwise), the region in the subject's retina in which the retinal lesion is located, as disclosed in detail hereinafter. It is noted that the storage device or memory used for storing the test results data may be included in or suitably linked or coupled to the computer system 105 or the computer 110, or the server 140. Alternatively, the storage device may be a shared device which is shared by or accessible to one or more of the computer system 105 or the computer 110, or the server 140, over a communication network. Thus, while the test results data may be stored locally on the system 105, this is not obligatory, and the test results may be stored elsewhere as disclosed hereinabove.

In step 380 it is determined whether adequate mapping of the field of vision was achieved. For example, it may be checked whether the number of lines 522 presented to the subject is less than a predetermined number, such as, for example, 40 (or any other suitable predetermined number). If the number of lines 522 presented to the subject is less than the predetermined number, the process may return to step 350 and a new line 522 is presented to the subject. Steps 350 to 380 may be repeated several times, for example 40 times (or any other suitable predetermined number of times suitable for such a test). In each repetition the line 532 may be presented at a different location of the screen 112 until the region of the subject's macular visual field has been appropriately mapped.

It is noted that such mapping may be achieved in more than one way. For example, in accordance with one preferred embodiment, after the line 532 is presented or displayed to the subject 101, and the subject has finished marking the segments that appeared different than the corresponding segments of the line 522, or alternatively to mark the segments that appeared different than the “virtual” predetermined reference pattern (a straight segmented line mentally conceived by the subject), the subject may visually fixate the tested eye on a fixation target 428A (see screens 530, 540, 550 and 560) in the vicinity of the line 532, by bringing the cursor 425 to point at the fixation target 428A and clicking a button on the mouse 125 to indicate fixation as disclosed in detail hereinabove. This may trigger the repeating of steps 365 and 370, which will result in the disappearing of the line 522 and the showing of a new line (not shown) at a new position on the screen 112 which is different than the position at which the line 532 was previously presented. The subject may then proceed to mark any segments at which a difference was perceived as disclosed hereinabove. The presentation may be similarly continued until adequate mapping of the field of vision has been performed.

Alternatively, in accordance with another embodiment, after the line 532 is presented or displayed to the subject 101, and the subject has finished marking the segments that appeared different than the corresponding segments of the line 522, or alternatively to mark the segments that appeared different than the “virtual” predetermined reference pattern (a straight segmented line mentally conceived by the subject), the line 532 may be caused to disappear from the screen 112, and the line 522 and the fixation target 428 may be again presented to the subject 110 in the same positions illustrated in screen 520. The subject may then again fixate on the fixation target 428 by bringing the cursor 425 to point at the fixation target 428 and click the mouse 125 to indicate fixation. The computer 100 may then present a new test pattern (not shown) at another new position relative to the position of the line 522 and the process may repeat after the subject marked any segments for which a difference was observed. By randomly or pseudo-randomly selecting a new line position for each new repetition the process may thus achieve adequate mapping of the desired macular area.

It is noted that if the first test pattern (such as for example the straight segmented line 522) which is presented to the subject happens to be projected on a region of the retina which is lesioned, the subject 101 may initially perceive the pattern to be distorted or modified but after a certain time the test pattern may be perceived to be identical with the predetermined reference pattern (such as for example a straight non-distorted segmented line due to the “filling in” phenomenon disclosed hereinabove. In such a case, the subject 101 may indicate or mark the location of the initially perceived distorted or modified region or component of the first test pattern, by using the mouse 125 and the cursor 425 as disclosed hereinabove. Alternatively, the subject 101 may be instructed (before or during the test) to ignore the initially perceived distortion or modification and to proceed to perform the fixation on the fixation target 428 as disclosed hereinabove by bringing the cursor 425 to point at the fixation target 428 and clicking a button on the mouse 125. When the second test pattern, such as the line 532 is then presented at another location on the screen 112 (see screen 530 of FIG. 5), the subject 101 may temporarily perceive a modification or distortion as the subject 101 shifts his vision from the fixation target towards the location of newly presented test pattern (which in this example is the location of the line 532 of screen 530 of FIG. 5). Therefore, in such cases in which the image of the first test pattern falls on a lesioned retinal area, the subject may perceive a distortion or modification in each of the repetitions or iterations of the test, irrespective of the location at which the second test pattern is presented on the screen 112. The distortion will be perceived after the patient shifted his vision from the fixation target towards the test pattern presented at the new location due to the fact that the shifting causes the image of the newly presented test pattern to be projected on the lesioned retinal area. Because of this phenomenon, the subject may mark a distortion or modification on all the second test pattern repetitions, and all of the marked distortions will tend to be marked at positions along the test pattern which approximately correspond to the position of the distortion or modification which was initially perceived at the first time of presentation of the first test pattern due to the presence of the retinal lesion.

If the test results do exhibit such an approximate “alignment” of multiple markings of perceived distortions or modifications at approximately similar positions on the test pattern, irrespective of the location of the presented second test patterns, this may be taken as an indication that there is at least one suspected retinal lesion at a position in the retina on which the image of first test pattern was projected.

While it is possible to perform the testing by mapping the field of view of the patient using only horizontal line patterns (such as the line 522) and moving the horizontal line patterns vertically to different positions on the screen 112, the mapping may also be performed using vertical lines (not shown in FIG. 5) that may be moved horizontally to different positions on the screen 112. It may also be possible to use a plurality of orthogonal horizontal and vertical lines in the same mapping test, in which case the mapping coverage of the field of vision may resemble a grid of intersecting lines (not shown).

The mapping of the field of view may also be done using a series of lines that are inclined at an angle to the horizontal or vertical orientation (slanted lines), or combination of series of slanted lines which may intersect each other either orthogonally or non-orthogonally, such that if these lines were all displayed at the same time on the screen 112 they may form a grid of intersecting lines (not shown).

In step 380 it is checked whether adequate mapping of the field of vision of the tested eye has been achieved. For example, if the location of presentation of the test pattern is different at each repetition or iteration of the test, adequate mapping may be ensured by checking that the number of lines presented to the subject has reached a predetermined number of iterations ensuring that data has been collected which suitably covers or maps the entire field of vision at a desired resolution.

Other different methods may however also be used to check adequate mapping. For example, if the testing of each location needs to be repeated more than one time and the location of presentation of test patterns is randomly or pseudo-randomly selected, the locations of performed tests may be compared with a look-up table to verify that the desired number of test repetitions for each test pattern location has been performed. If adequate mapping has not been achieved the process may return control to step 350 to present the next test pattern.

If adequate mapping of the field of vision of an eye has been achieved, the process may proceed by determining whether only one eye has been examined so far (step 382). If only one eye has been examined, the subject may be instructed to uncover the non-examined eye and to cover the examined eye (step 385). The process may then return to step 350 with the subject testing his other eye as disclosed in detail hereinabove. If both eyes have been examined, the process may terminate (step 390).

The position of the line 522 presented or displayed to the subject on the screen 112 may thus be varied in order to appropriately cover the macular area at a desired resolution so as to detect lesioned retinal regions. It is noted that in accordance with one preferred embodiment, the mapping may be performed more than once, and that the central part or foveal region of the macular area of the retina may also be mapped at a higher resolution than the rest of the macular area. This may be accomplished by presenting to the subject test patterns such as the line 522 at locations which are relatively close to one another on the screen 112. This may result in higher lesion mapping resolution in the foveal region.

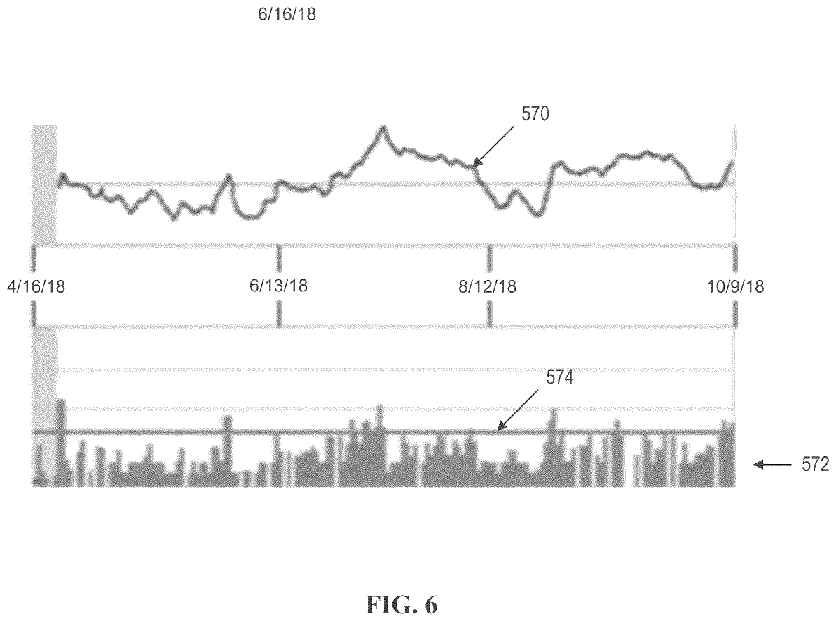

FIG. 6 shows a plot 570 and bar chart 572 of example visual field test scores for a sequence of visual field tests of the method 300 relative to a screening threshold 574. The screening threshold 574 can be selected using any suitable approach, such as the approaches described herein, to trigger a change in monitoring of a patient's eye for the development of a retinal condition (e.g., non-exudative neovascularization, wet (exudative neovascular) AMD, retinal atrophy, central geographic atrophy, and/or non-central geographic atrophy) from repeated visual field tests to repeated imaging examinations.

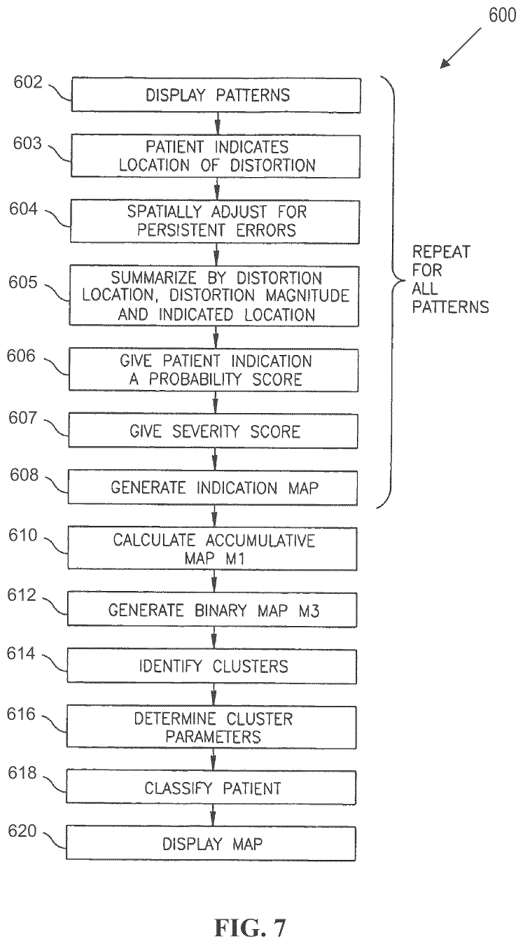

FIG. 7 is a simplified schematic flow chart diagram of a method 600 for mapping an eye using visual field tests to detect increased risk for development of a retinal condition (e.g., non-exudative neovascularization, wet (exudative neovascular) AMD, retinal atrophy, central geographic atrophy, and/or non-central geographic atrophy), in accordance with embodiments. Optionally, the patient performs the test procedure with one eye covered. In some embodiments, after a first eye is tested, the second eye is tested, if necessary. Additional details of the method 600 is described in U.S. Pat. No. 7,665,847, which is hereby incorporated herein by reference in its entirety.

In each test session, a plurality of patterns with a distortion are displayed (act 602) on screen 112. In some embodiments, the stimuli are displayed for short durations up to about 400 milliseconds, for example between about 100-200 milliseconds. For each displayed pattern, the patient is requested to indicate (act 603) the location of the distortion. The results for each pattern are optionally summarized (act 605) by the location of the distortion, the magnitude of the distortion and the location indicated by the patient. In some embodiments, the location indications are spatially adjusted (act 604) in order to correct for persistent errors in the patient's pointing, perception and/or other non-eye mechanisms. In some embodiments, a record is kept of distortions for which no response was received. The number of distortions for which patient indications were not received is optionally used in determining the reliability of the test. Alternatively or additionally, distortions for which patient indications are not received are used in evaluating the areas of the displayed distortion as the distortions may not have been perceived by the patient due to a lesion, overlying the displayed distortion, on the patient's visual field.

Each patient indication is given (act 606) a probability score indicative of the probability that the patient indication is indicative of impaired eye tissue and is not a correct indication of the location of the displayed distortion. Generally, if the patient indicates a position close to the actual distortion, the patient's indication is most probably indicative of healthy eye tissue as the patient identified the displayed distortion. If, however, the patient indicates a different position, the patient's eye may be impaired at the indicated location and therefore the patient did not identify the displayed distortion, which was overridden by a competing pathological stimulus caused by a retinal-related lesion. The patient indication is given (act 607) a severity score S, optionally as a function of the distortion size and the probability score. As the distortion is greater, the retinal lesion needs to be more severe in order to cause the patient to point in the wrong location.

For each displayed pattern, a map M2 indicative of the health of each point of the eye tissue of the patient based on the response of the patient to the displayed pattern, is generated (act 608). An accumulative map M1 is calculated (act 610) based on the maps M2 of each of the patterns. In some embodiments, a binary map M3, which indicates impaired areas on accumulative map M1, is generated (act 612). The binary map generally identifies (act 614) clusters of impaired eye tissue. Parameters of the impaired clusters are optionally determined (act 616) and the patient is classified (act 618) according to the cluster parameters. Alternatively or additionally, accumulative map M1 and/or a clustered variation thereof is displayed (act 620) for analysis.

Referring in detail to displaying (act 602) patterns on the screen, in some embodiments, in order to map the patient's visual field, patterns having distortions at different areas on the visual field are displayed to the patient. In addition, patterns with different magnitude of distortion are displayed. Each pattern is optionally represented by the location of the distortion relative to the center of the display (corresponding to the fovea) and the magnitude of the distortion.

Alternatively or additionally to displaying (act 602) patterns with distortions, patterns without distortions are displayed. Distortions detected by the patient in this alternative are due to imperfections in the patient's visual field, as no distortions were displayed.

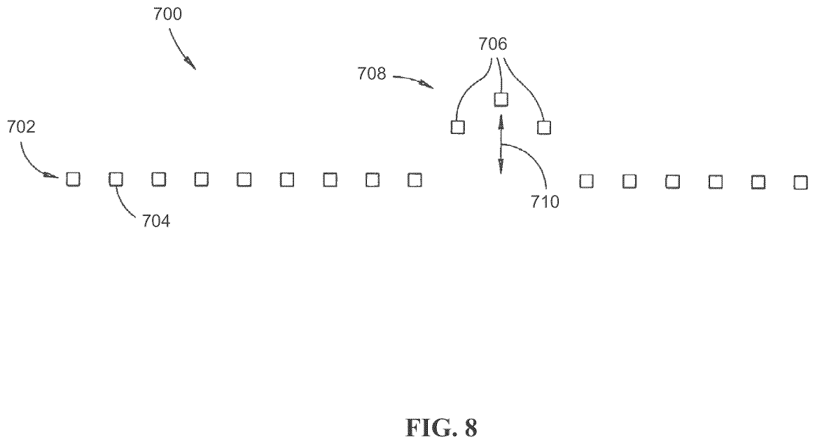

FIG. 8 is a schematic illustration of a pattern format of the patterns displayed to a patient by the system 100 in the method 600. Pattern format 700 comprises a fragmented line 702 formed of a plurality of squares 704, for example between about 10-30 squares (e.g., 27-29). Alternatively, the line 702 has fewer than 10 squares or more than 30 squares. Further alternatively, a continuous line is used. One or more squares 706 (3 squares in FIG. 8) are displaced from line 702, so as to form a distortion 708.

Line 702 optionally has a length which covers between about 10-20° (e.g., 14°) of the patient's visual field, when the patient is situated at a normal distance from screen 112, for example about 30-60 centimeters. It is noted, however, that the tests are valid even if the patient sits much closer to screen 112 or much farther from screen 112. In the following description, whenever a length or distance is stated in degrees it means that the length covers that angle on the patient's visual field under these conditions.

In different displayed patterns, line 702 is displaced from the center of the display, by different distances, so as to cover substantially the entire area covered by the display. In an exemplary embodiment, line 702 is displaced from the center of the display by different distances up to about −7.0° to 7.0°, depending on the size of the mapped visual field. In larger visual fields larger distances from the center may be used. It is also noted that non-symmetrical distances from the center may be mapped. The different patterns displayed to the patient optionally further differ in the distance (indicated by an arrow 710) between distortion 708 and line 702. The distances 710 of the different patterns are optionally between a maximal distance and a minimal distance. The maximal distance is optionally selected as a distance close to a value so large that it will be identified even by patients with severe lesions. Using larger distances will generally not add information on the patient's visual field. The minimal distance is optionally selected as a distance close to a distortion level that will not be identified by substantially all patients and therefore does not add information on the patient's visual field. In an exemplary embodiment, the distances 710 used are between about 0.1-0.35 degrees, although other distances may be used, including distances even up to about 0.8-2.0 degrees and more.

The different patterns optionally differ in their orientation. In an exemplary embodiment, in some of the pattern's line 702 is horizontal and in other patterns line 702 is vertical. In some embodiments, diagonal patterns are used, in addition to, and/or instead of, the vertical and horizontal patterns. The squares 706 forming distortion 708 are optionally curved in the direction opposite the fovea, such that the central square 706 is farther from the fovea than the side squares 706. This distortion direction is similar to the pathological distortion of AMD patients. Alternatively, other shaped distortions are used, including distortions in the direction of the fovea.

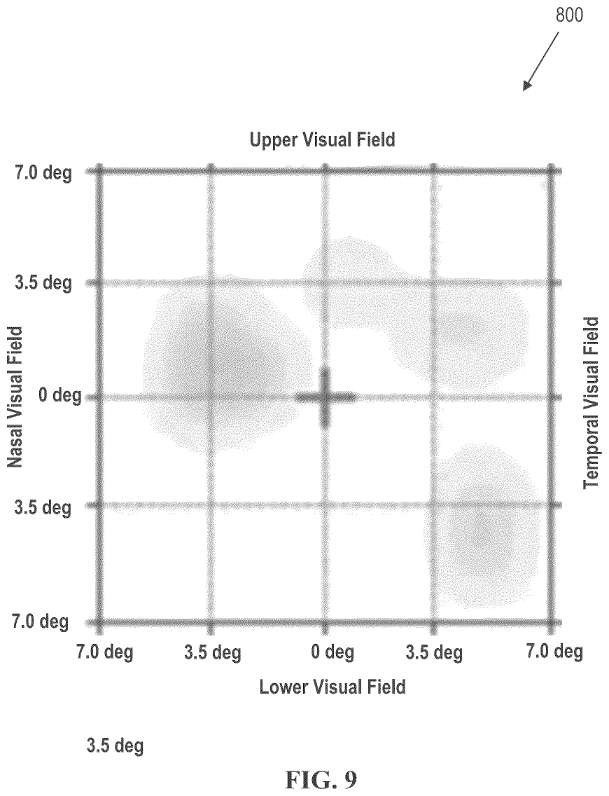

FIG. 9 is shows an example metamorphopsia map 800 of an eye generated using the method 600. The map 800 displays shaded contours indicative of the distribution of afflicted tissue as well as the extent of the affliction. The map 800 can be processed using any suitable approach to generate a visual field test score for comparison with a screening threshold. For example, the extent of the affliction across the eye can be integrated to determine the visual field test score for comparison with a screening threshold. The occurrence of a visual field test score that exceeds the screening threshold can be used to trigger a switch between monitoring the eye for the development of the retinal condition via repeated visual field tests and monitoring the eye via repeated imaging examinations of the retina.

### Screening Threshold and Increased Risk Level

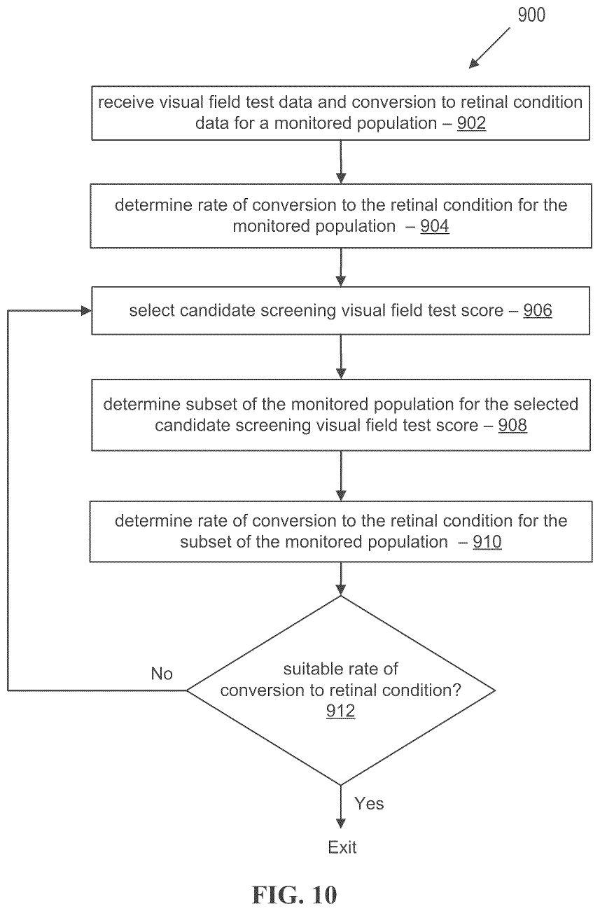

FIG. 10 is a simplified schematic flow chart diagram of a method 900 of determining a screening threshold visual field test score for use in the method 10. The method 900 includes receiving visual field test data and corresponding conversion a retinal condition (e.g., non-exudative neovascularization, wet (exudative neovascular) AMD, retinal atrophy, central geographic atrophy, and/or non-central geographic atrophy) data indicative of the timing of any subsequent conversion to the retinal condition in the monitored eye for a monitored population (act 902). In act 904, the data received in act 902 is analyzed to determine an overall rate of conversion to the retinal condition for the total monitored population. In act 906, a candidate screening visual field test score is selected. In act 908, a subset of the total monitored population is determined that has at least one visual field test score that equals or exceeds the candidate screening visual field test score. In act 910, a rate of conversion to the retinal condition for the subset of the total monitored population (determined in act 908) is determined. Any suitable approach can be used in acts 904, 910 to determine the rate of conversion to the retinal condition. For example, Kaplan-Meier survival analysis can be employed to calculate the risk of conversion to the retinal condition in acts 904, 910. In act 912, the rate of conversion to the retinal condition of the subset of the total monitored population (determined in act 908) is evaluated to determine whether to evaluate another candidate screening visual field test score via repeating of act 906 through 912.

### Example Results

Eyes diagnosed with intermediate dry AMD (iAMD) were at elevated risk for conversion to neovascular AMD (nAMD or wet AMD) with the highest risk observed in eyes with advanced AMD in the fellow eyes (FE) reaching 50% in 5 years (A Simplified Severity Scale for Age-Related Macular Degeneration: AREDS Report No. 18 Age-Related Eye Disease Study Research Group. Arch Ophthalmol. 2005 November; 123(11): 1570-1574. doi: 10.1001/archopht.123.11.1570). Recently available data from two prophylactic treatments studies that compared quarterly ranibizumab and aflibercept injections to a sham control indicate disappointing results after 2 years of follow up, with conversion rates of 10% and 13% (Intravitreal Aflibercept Injection vs Sham as Prophylaxis Against Conversion to Exudative Age-Related Macular Degeneration in High-risk Eyes. A Randomized Clinical Trial. Jeffrey S. Heier, David M. Brown, Sumit P. Shah et al. JAMA Ophthalmol. 2021; 139(5):542-547. doi: 10.1001/jamaophthalmol.2021.0221) and (Prophylactic Ranibizumab to Prevent Neovascular Age-Related Macular Degeneration in Vulnerable Fellow Eyes: A Randomized Clinical Trial. Clement K. Chan, M D, Maziar Lalezary, M D, Prema Abraham, M D, et al. on behalf of the PREVENT Study Group. PII: S2468-6530(22)00041-0 DOI: URL//doi.org/10.1016/j.oret.2022.01.019).

Management of nAMD is entering an era of management regimens resulting in long inter-visit periods. Several studies with gene therapy, slow-release devices, and higher dosing of cleared drugs are ongoing (e.g., New and Innovative Treatments for Neovascular Age-Related Macular Degeneration (nAMD). Patel, P.; Sheth, V. J. Clin. Med. 2021, 10, 2436. URL//doi.org/10.3390/jcm10112436) and two treatment options were recently cleared by the FDA (New 2-year data for Genentech's Vabysmo and Susvimo reinforce potential to maintain vision with fewer treatments for people with two leading causes of vision loss. Genentech. News release. Feb. 10, 2022. Accessed Feb. 11, 2022. URL//www.gene.com/media/press-releases/14944/2022-02-10/new-two-year-data-for-genentechs-vabysmo). These drugs typically have a common objective of reducing the treatment burden while maintaining non-inferiority in visual and anatomical outcomes compared to the current treatments.

The increased follow-up intervals between visits may lead to an increase in the time before detection of CNV conversion in the fellow eye. The increase in time amplifies the unmet need for management strategies for these eyes.

In an at home randomized clinical trial, the ForeseeHome (Notal Vision Inc., Manassas, VA) was found useful for early detection of nAMD (AREDS Home Study Research Group. Randomized trial of a home monitoring system for early detection of choroidal neovascularization home monitoring of the Eye (HOME) study. Ophthalmology 2014; 121:535-44). The ALOFT study was a retrospective analysis of the 10 years visual acuity outcome data of 2123 patients participating in the ForseeHome program with excellent long term outcomes (Analysis of the Long-term visual Outcomes of ForeseeHome Remote Telemonitoring—The ALOFT study. Mariam Mathai, Shivani Reddy, Michael J. Elman, Richard A. Garfinkel, Byron Ladd, Alan Wagner, George E. Sanborn, Jennifer Jacobs, Miguel Busquets, Emily Y. Chew on behalf of the ALOFT study group. Ophthalmology Retina: DOI: URL//doi.org/10.1016/j.oret.2022.04.016).

During the analysis of the ALOFT data, eyes that had a false positive (FP) alert while monitoring at home were labeled by the AMD status of the fellow eye (i.e., iAMD vs nAMD). The analysis of the ALOFT data investigated the hypothesis that based on the outcomes of an alert and the status of the FE, home monitoring can identify a subgroup of eyes that are at an elevated risk for future conversion.

A retrospective review was conducted of records (from August 2010 to July 2020) of all dry AMD patients from five high referring clinics. The review included all home monitored eyes that had a ForeseeHome alert (i.e., had a visual field test score that exceeded a threshold), and conversion to non-exudative neovascularization and wet AMD was not detected within one month from the alert (i.e., a False Positive (FP)). For each of these eyes, the dry AMD vs wet AMD status of the fellow eye at enrollment was recorded. The eventual outcomes, including date of detection of conversion to non-exudative neovascularization and wet AMD by the clinician and initiation of treatment for wet AMD (which may or may not be while still monitored at home) or the date of the study final follow up visit without a conversion to non-exudative neovascularization and wet AMD were recorded. The recorded data was used to calculate Kaplan-Meier (K-M) survival functions describing the risk for conversion over time after a FP by the AMD status of the fellow eye. Reference data was collected from all the ALOFT study eyes upon enrollment and from the PROCON (Intravitreal Aflibercept Injection vs Sham as Prophylaxis Against Conversion to Exudative Age-Related Macular Degeneration in High-risk Eyes. A Randomized Clinical Trial. Jeffrey S. Heier, David M. Brown, Sumit P. Shah et al. JAMA Ophthalmol. 2021; 139(5):542-547. doi: 10.1001/jamaophthalmol.2021.0221) and PREVENT (Prophylactic Ranibizumab to Prevent Neovascular Age-Related Macular Degeneration in Vulnerable Fellow Eyes: A Randomized Clinical Trial. Clement K. Chan, M D, Maziar Lalezary, MD, Prema Abraham, MD, et al. on behalf of the PREVENT Study Group. PII: S2468-6530(22)00041-0 DOI: URL//doi.org/10.1016/j.oret.2022.01.019) studies that were designed to enroll eyes at high risk for conversion. A Log-Rank test for the K-M survival functions was used to compare ALOFT FP nAMD FE to each of the other groups.

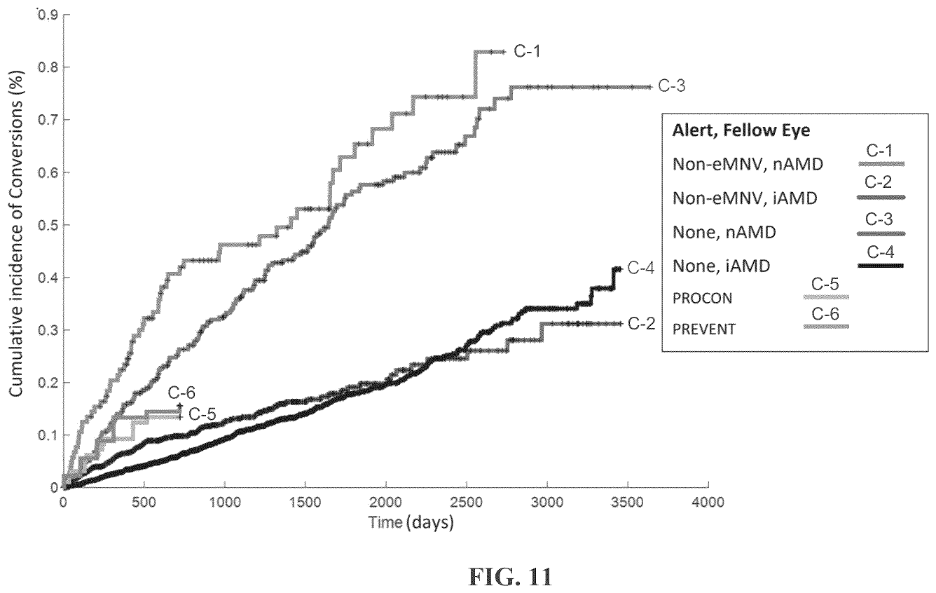

The ALOFT study included 3334 eyes of 2123 participants that were monitored with the home visual field test device. A total of 953 (29%) of the eyes had a False Positive. Of the 953 eyes having a False Positive, 106 were fellow eyes to a wet AMD eye. 56 (53%) of the 106 eyes developed wet AMD. 847 of the 953 eyes were fellow eyes to a dry AMD eye. 110 (13%) of the 847 eyes developed wet AMD. The balance of the 953 eyes (787 eyes) did not convert by the time of data collection. From time establishing a baseline (BL) of 3334 eyes, 151 of 303 eyes (50%) with a wet AMD fellow eye and 388 of 3001 eyes (13%) with a dry AMD fellow eye converted. In the PROCON study, 128 eyes were enrolled. Of the 97 eyes that completed the study, 13 eyes, of which 7 were in the sham arm and 6 in the prophylactic treatment arm converted during the study and 84 eyes did not convert, i.e. censored after 24 months. The approximate timing of conversions was reconstructed from the published K-M graph. In the PREVENT study, 108 eyes were enrolled. Of the 90 eyes that completed 24 months follow up, 14 eyes, of which 7 were in the sham arm and 7 in the prophylactic treatment arm converted during the study and 76 eyes did not convert, i.e. censored after 24 months. Study visits took place every 3 months, and the 14 conversions were associated with these visits, allowing for inclusion in the analysis. Since the event rates for both arms of both studies were not different, they were pooled together in each study for this analysis. Eyes that dropped out during these studies were conservatively assumed as excluded at enrollment, therefore providing a slightly overestimated rate of conversion. The K-M estimate of conversion functions for the 6 groups are presented in FIG. 11. A Log-Rank test comparing the FP with wet AMD fellow eye to FP with dry AMD fellow eye, base-line wet AMD fellow eye, base-line dry AMD fellow eye (all from ALOFT, PROCON and PREVENT) showed a significant difference in the K-M conversion function, p<106, p=0.04449, p<106, p=0.00004 and p=0.00028 respectively. See supplemental material Table 1 (available at URL www.ophthalmologyretina.org/) with the conversion rates at various time points for the 6 groups.

All eyes monitored by ForeseeHome that are fellow to eyes diagnosed with wet AMD are at very high risk for future conversion. The predictive value of false positive alerts for risk of future conversion in eyes with a wet AMD fellow eye was found to be significantly higher compared to eyes in other subgroups of ALOFT or in comparator trials. The observation that wet AMD was not identified with standard care examination immediately after the alert can have several explanations. To mention few, the false positive alert may be a result of other changes beneath the retinal pigment epithelium (RPE) including fluid, drusenoid pigment epithelial detachments (PED) and fibrovascular PED or nonexudative macular neovascularization resulting in change in the functional hyperacuity test score that triggered the alert while no manifestation of exudation is present. It is also possible that in some rare cases the interpretation of spectral domain optical coherence tomography (SD-OCT) images in routine care setting missed a conversion, which might have been identified by a reading center or if optical coherence tomography angiography (OCT-A) or fluorescein angiography (FA) were performed.

FIG. 11 shows Kaplan-Meier estimated conversion functions for the ALOFT, PROCON, and PREVENT studies. The abbreviations employed in the legend of FIG. 12 include:
FP nAMD FE=Eyes fellow to nAMD after a false positive alert;
FP iAMD FE=Eyes fellow to iAMD after a false positive alert;
BL nAMD FE=Eyes fellow to nAMD from baseline; and
BL iAMD FE=Eyes fellow to iAMD from baseline alert.

Other variations are within the spirit of the present invention. Thus, while the invention is susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

1. A method of identifying a patient at increased risk for development of a retinal condition of a retina of the patient, the method comprising:
monitoring the patient via one or more visual field tests, wherein each of the visual field tests comprises displaying stimuli for viewing by an eye of the patient, receiving indications of locations of the stimuli as perceived by the patient, and determining a visual field test score based on the stimuli and the indications of locations of the stimuli as perceived by the patient; and
in response to at least one of the visual field test scores exceeding a screening threshold, identifying the patient as being at increased risk for development of the retinal condition relative to a selected population.

2. The method of claim 1, wherein the retinal condition comprises wet age-related macular degeneration (AMD).

3. The method of claim 1, wherein the retinal condition comprises retinal atrophy.

4. The method of claim 1, wherein the retinal condition comprises central geographic atrophy.

5. The method of claim 1, wherein the retinal condition comprises non-central geographic atrophy.

6. The method of claim 1, further comprising monitoring the patient via at least one imaging examination of the retina in response to the at least one of the visual field test scores exceeding a screening threshold.

7. The method of claim 6, wherein development of the retinal condition is not detected during a subsequent examination of the at least one imaging examination of the retina.

8. (canceled)

9. The method of claim 6, wherein the at least one imaging examination comprises an optical coherence tomography (OCT) examination of the retina.

10. The method of claim 6, wherein the at least one imaging examination comprises an optical coherence tomography angiography (OCTA) examination of the retina for development of non-exudative neovascularization.

11. The method of claim 6, wherein the at least one imaging examination comprises a dye-based angiography.

12. The method of claim 11, wherein the dye-based angiography comprises fluorescein angiography.

13. The method of claim 11, wherein the dye-based angiography comprises indocyanine green angiography.

14. The method of claim 6, wherein:
the one or more visual field tests are conducted at a first frequency; and
the at least one imaging examination comprises a sequence of imaging examinations conducted at a second frequency that is greater than the first frequency.

15. The method of claim 14, wherein the second frequency is at least twice the first frequency.

16. The method of claim 1, wherein development of the retinal condition is not detected within one month of one of the visual field test scores exceeding the screening threshold.

17. The method of claim 1, wherein each of the one or more visual field tests measures visual hyperacuity.

18. The method of claim 1, wherein the screening threshold is selected so that the patient has at least a 50 percent increase in risk of development of the retinal condition relative to the selected population.

19. The method of claim 18, wherein the screening threshold is selected so that the patient has at least a 100 percent increase in risk of development of the retinal condition relative to the selected population.

20. (canceled)

21. The method of claim 1, wherein a contra-lateral eye of the patient comprises wet AMD.

22. The method of claim 1, wherein the increased risk for development of the retinal condition is for a time range between 1 month and 60 months from exceedance of the screening threshold.

23.-35. (canceled)
